(19) United States
(12) Patent Application Publication
(10) Pub. No.: US 2011/0125061 A1
(43) Pub. Date: May 26, 2011

(54) Monitoring Conditions Of A Patient's Urinary System

(75) Inventors: Menashe Shahar, Hagolan (IL); Ori Sahar, Hagolan (IL)

(73) Assignee: P SQUARE MEDICAL LTD., Herzliya Pituach (IL)

(21) Appl. No.: 12/991,948

(22) PCT Filed: May 13, 2009
(86) PCT No.: PCT/IL2009/000479

§ 371 Date: Feb. 3, 2011

Related U.S. Application Data
(63) Continuation-in-part of application No. 12/119,921, filed on May 13, 2008.
Application No. 12/119,921 is a continuation-in-part of application No. PCT/IL2006/001463, filed on Dec. 20, 2006.

(51) Int. Cl. A61B 7/00

(52) U.S. Cl. 600/586

(57) ABSTRACT

A monitoring system and method are presented for use in monitoring a condition of a patient's urinary system. The monitoring system comprises an acoustic assembly comprising at least one acoustic receiver adapted for receiving acoustic signals during a patient's urination and generating data indicative thereof. The monitoring system also includes a control unit that is in communication with said acoustic assembly. The control unit is configured and operable for analyzing said generated data indicative of the continuously received acoustic signals during a patient's urination, obtaining a time variation of the acoustic signal during the urination and determining a corresponding spectral data of the acoustic signal. The control unit further analyzes the spectral data and, upon detecting at least one first signal peak corresponding to a condition of turbulence in the urine flow, determining a relation between said first signal peak and a second signal peak corresponding to a condition of laminar urine flow. Based on said relation, the control unit determines the condition of a patient's low urinary system and generating output data indicative thereof.

### CROSS-REFERENCE TO RELATED APPLICATIONS

This Application is a US National Phase of International Application PCT/IL2009/000479 filed on May 13, 2009, which in turn claims priority to U.S. application Ser. No. 12/119,921 filed on May 13, 2008, both of which are incorporated herein in their entirety.

### FIELD OF THE INVENTION

This invention is generally in the field of medical devices, and relates to a device and method for monitoring conditions of a patient's urinary system.

### BACKGROUND OF THE INVENTION

Monitoring of a condition of a patient's urinary system is needed for detecting various types of the urine system abnormality, including inter alia prostate enlargement. The latter is a widespread phenomenon developed in more than half men over age 50. By age 80, about 80% of men have enlarged prostates. The prostate enlargement is thought to be related to hormonal disorders typical to the age, and is termed Benign Prostatic Hyperplasia or BPH. In a minority of the cases, the prostate enlargement involves prostate cancer.

Whatsoever be the cause, enlarged prostate may lead to bladder control problems. This is because the prostate gland encircles the urethra beneath the bladder neck. An enlarged prostate exerts pressure on the urethra which may deform its shape and reduce its cross sectional area. In acute circumstances, a total obstruction of the urethra might occur.

A quantitative diagnosis of the urethral condition, such as urethral obstruction, can help in early detection of prostate problems, which in turn allows for anticipating medication or other appropriate treatment. In cases where bladder control problems exist already, a quantitative diagnosis may help in determining severity of the case and in monitoring the effect of the treatment procedures been taken.

From a broader perspective, a quantitative diagnosis of urethral obstruction is only one of several common tests taken during the somewhat complicated process of screening and diagnosing for Lower Urinary Tract Symptoms (LUTS). Lower Urinary Tract Symptoms may involve several factors, including disorders in the somatic nervous system, in the bladder/urethral autonomic nervous system, in the detrusor and in the sphincter muscles, and more. Said screening process is therefore a must for distinguishing between the plurality of medical situations that may cause a patient to experience urinary problems.

Facilitating and simplifying the recognition and the quantitative diagnosis of urethral condition may therefore be essential not only in case an obstruction does exist, but also in negating its existence in the opposite case thus leading toward a correct diagnosis.

Methods commonly used for quantitative detection of urethral and prostate conditions include the following techniques: a digital rectal exam to feel for prostate enlargement; cystoscopy (under local anesthetic) consisting of passing a lens into the urethra and bladder to examine if any abnormalities are present; intravenous pyelogram consisting of X-ray irradiation of the urinary tract as a dye is injected into a vein that shows up tumors or obstructions; transrectal ultrasonography that uses a rectal probe for assessing the prostate; transabdominal ultrasonography that uses a device placed over the abdomen; and urodynamic technique including measurements of a urine flow speed (uroflowmetry) for quantitative detection of urethral, bladder and prostate conditions.

Various techniques have been developed, for determining the prostate related conditions, based on acoustic methods. These techniques are disclosed for example in the following patents and patent publications: U.S. Pat. No. 6,063,043; U.S. Pat. No. 6,428,479; WO 05/067392; WO 05/004726; and RU 2224464.

WO 07/072,484, by the inventors of the present application, discloses a system and method for the determination of urethral blockage, utilizing a transducer arrangement for locating in the vicinity of the patient's urine flow, and a control unit in communication with the transducer arrangement. The transducer arrangement has at least one acoustic transducer capable of at least receiving acoustic waves, generated by the patient's urine flow, and producing an output signal indicative thereof. The control unit receives and processes the output signal and determines a change in the output signal indicative of the urethral blockage.

General Description

There is a need in the art for a novel technique for non-invasive instant indication of parameters indicative of a patient's urinary system condition (in particular, urinal flow velocity profile, urethral obstruction degree, pressure in urinary bladder and detrusor pressure), capable of shortening and facilitating the process of screening and diagnosing for Lower Urinary Tract Symptoms (LUTS), even before any physical symptoms have actually been experienced by the patient.

The currently used non-invasive methods are practically incapable of determining an urethral obstruction or performing a quantitative measurement thereof. For example, uroflowmetry is practically incapable of determining urethral obstructions and/or their severities unless the internal bladder pressure is also known. Also this conventional technique cannot determine the cause of obstruction, which can be due not only to BPH, but possibly also to abnormalities in the urethra, weak bladder muscles, or other causes. This is because on the one hand a low urinary flow rate may be an indication of a detrusor problem rather than of an urethral obstruction, while on the other hand a normally detected flow rate should not necessarily indicate of a normal urethra since it may result from extra abdominal/bladder pressures compensating against certain flow resistance caused by urethral obstruction. Uroflowmetry combined with simultaneous measurement of internal bladder pressure is thus required in order to allow for discrimination between the different factors (i.e. the urethra flow resistance and the abdominal/bladder pressure). Internal bladder pressure measurement involves however invasive procedure—inserting a catheter into the bladder. The inconvenience and infection risks accompanied to the procedure make its use rare and appropriate for special cases only.

The present invention takes advantage of the fact that a urine flow generates acoustic signals of unique Strouhal frequencies, and provides a novel technique based on measuring and analyzing these acoustic signals and extracting data indicative of various parameters characterizing the patient's urinary system condition. These parameters include an urination time, amount of urinated urine, urinal flow velocity profile (time function of the velocity), urinary flow rate; urethral obstruction degree, pressure in urinary bladder and detrusor pressure from a patient. In particular, the urethral obstruction causes a turbulent-like urine flow through the urethra, which is of a differing nature than that of laminar-like urine flow in a non-obstructed urethral part. The inventors have found that such a turbulence-like flow of the urine generates additional acoustic signals in the Strouhal frequencies' range. Accordingly, the recognition of signals typical to a turbulent-like flow is indicative of the obstructed urine flow through the urethra, the frequency and magnitude of which may be indicative of the severity of the obstruction and a location of the obstruction (its distance between the transducer interface).

It should be understood that the expressions “turbulent-like” or “turbulent” and “laminar-like” or “laminar” used herein to describe the urine flow behavior, refer to the urine flows which, while being not absolutely turbulent and laminar, differ from one another towards respectively turbulence and laminar behavior of the flow.

According to the invention, acoustic signals are (continuously or quasi continuously) detected and optionally sampled during the patient's urination, and data indicative of these acoustic signals is analyzed. The data analysis comprises determination of spectral data indicative of the so-detected acoustic signals, and further analysis of this spectral data to identify whether the spectral data includes at least one first signal peak within a certain frequency range corresponding to a condition of turbulence in the urine flow. In case such a first peak exists, a relation between this first signal peak and a second signal peak within a different frequency range corresponding to a condition of laminar urine flow is determined. Based on the so-determined relation, the condition of a patient's urinary system can be determined and output data indicative thereof can be generated.

The present invention, according to its one broad aspect, provides a monitoring system for use in monitoring a condition of a patient's urinary system. The monitoring system comprises at least one acoustic receiver adapted for continuously detecting acoustic signals during the patient's urination and generating data indicative thereof; and a control unit in communication with the acoustic receiver(s). The control unit is configured and operable for analyzing said generated data. This is aimed at determining spectral data indicative of the acoustic-signals data, and further analyzing the spectral data. Upon detecting at least one first signal peak corresponding to a condition of turbulence in the urine flow, a relation between said first signal peak and a second signal peak corresponding to a condition of laminar urine flow. Based on said relation, the condition of a patient's urinary system can be determined and output data indicative thereof can be generated.

When desired, the monitoring system can comprise a positioning unit for positioning said at least one acoustic receiver in the vicinity of the patient's urine flow such that an acoustic interface of the receiver is in a position for receiving acoustic signals generated during the patient's urination.

According to some embodiments of the present invention, the spectral data includes a Strouhal frequency range which can be determined in a frequency range of about 20-1000 Hz. The first signal peak corresponding to the turbulent urine flow can be detected in a frequency range of 150-1000 Hz. The second signal peak corresponding to condition of laminar urine flow can be detected in a frequency range of 70-150 Hz.

According to an embodiment of the present invention, the control unit is configured and operable for analyzing the spectral data by determining a time variation of the relation between the first and second signal peaks, the first signal peak varying with time during the urination. More specifically, both peaks move towards higher frequencies when the flow becomes to be stronger. The time variation may be used to indicate the appearance of the maximal flow rate condition, which may in turn be utilized for optimal identification of peaks of the acoustic signals that are to be used for calculations.

When desired, the control unit is configured and operable to determine the relation between the first and second signal peaks by calculating at least one of the following: a ratio between amplitudes of the first and second signals (generally the amplitude profile of the measure signals), and a ratio between frequencies of the first and second signals, and time variations of these ratios during urination and/or during successive urinations. In operation, the control unit is configured and operable to calculate or estimate also one or more following parameters indicative of the urinary system condition: amount of urinated urine during the urination time, urinal flow velocity profile, urinary flow rate; urethral obstruction degree, urethral flow resistance, pressure in urinary bladder and detrusor pressure.

According to an embodiment of the present invention, the control unit comprises a memory utility for storing reference data comprising a given value or a range of values for at least one of the following parameters: an urethral diameter, urethral length, and elasticity of an urethral wall. When desired, the control unit can be also configured and operable to apply a predetermined model to the spectral data. This model can be based on a given value or a range of values for one or more of the above-defined parameters.

The present invention, according to another broad aspect, provides a method for use in monitoring a condition of a patient's urinary system. The method comprises detecting acoustic signals originated by urine flow during the patient's urination, and generating data indicative thereof. These data generated during the urination are analyzed and corresponding spectral data is generated and analyzed to thereby, upon detecting at least one first signal peak corresponding to a condition of turbulence in the urine flow, determine a relation between said signal peak corresponding to the condition of turbulence in the urine flow and a second signal peak corresponding to a condition of laminar urine flow. Using said relation, the condition of a patient's urinary system is determined and output data indicative thereof is generated.

According to some embodiments of the present invention, this continuous detecting the acoustic signals can be carried out by one or more acoustic receivers.

According to one embodiment of the present invention, there is provided a diagnostic kit for use in monitoring a condition of a patient's urinary system that is configured and operable according the above-described method.

According to another general aspect of the present invention, there is provided a method for use in monitoring a condition of a patient's urinary system. The method comprises analyzing spectral data corresponding to acoustic signals originated by urine flow during the patient's urination; and upon detecting at least one first signal peak corresponding to a condition of turbulence in the urine flow, determining a relation between said first signal peak and a second signal peak corresponding to a condition of laminar urine flow; using said relation to determine the condition of a patient's urinary system and generate output data indicative thereof.

According to yet another general aspect of the present invention, there is provided a computer system adapted for receiving data indicative of a sequence time and date of acoustic signals. This computer system is configured and operable for processing said data to determine spectral data indicative thereof, analyzing the spectral data and, upon detecting at least one first signal peak corresponding to a condition of turbulence in the urine flow, determining a relation between said first signal peak and a second signal peak corresponding to a condition of laminar urine flow. Based on said relation, the computer system generates output data indicative of a condition of a patient's urinary system from which said acoustic signals have been originated.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a block diagram of an example of a monitoring system of the present invention for monitoring a condition of a patient's urinary system;

FIG. 2 is an example of the configuration and operation of the monitoring system of FIG. 1;

FIG. 3A is a flow diagram of an example of a method of the present invention for use in the determination of the condition of a patient's urinary system;

FIGS. 3B and 3C are graphical representations of spectral intensity distributions of acoustic signals corresponding to normal (non-obstructed) and abnormal (obstructed) conditions of a urinary system respectively;

FIG. 4A is an example of quantitative measurement of sound urine flow using the method of the present invention;

FIG. 4B is an example of quantitative measurement of sound urine flow by the conventional uroflowmeter; and

FIG. 5 is a graphical representation of the experimental results of using the method of the present invention for the spectral analysis of Strouhal frequency ranges as function of the urine flow in patients with diagnosed bladder outlet (urethral) obstructions and in patients from a control group (with no bladder outlet obstructions).

### DETAILED DESCRIPTION OF EMBODIMENTS

The principles of the technique of the present invention may be better understood with reference to the drawings and the accompanying description, wherein like reference numerals have been used throughout to designate identical elements. It should be understood that these drawings, which are not necessarily to scale, are given for illustrative purposes only, and are not intended to limit the scope of the invention.

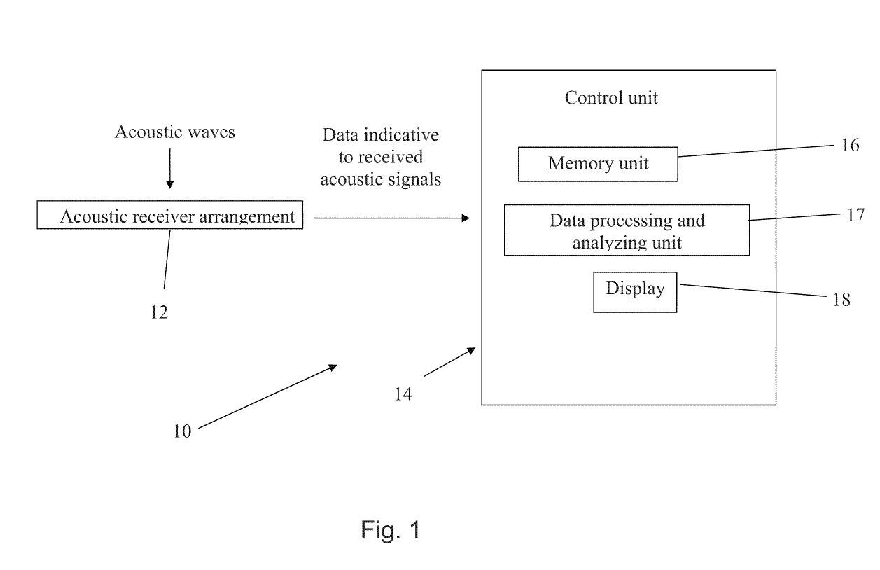

Referring to FIG. 1, there is illustrated, by way of a block diagram, a monitoring system 10 of the present invention for monitoring a condition of a patient's urinary system. The monitoring system 10 includes an acoustic assembly 12 including one or more acoustic elements that is/are capable of at least receiving acoustic waves and generating data indicative thereof, and a control unit 14 that is configured and operable for receiving and analyzing the data indicative to the acoustic waves received by the acoustic receiver assembly 12. The connection between the acoustic receiver assembly 12 and the control unit 14 is provided via wires or wireless signal transmission. In the latter case, the acoustic receiver assembly and the control unit are appropriately provided with communication utilities for transmitting and receiving IR, RF or acoustic data signals.

As indicated above, the acoustic receiver assembly 12 includes one or more acoustic receivers. These may be microphones or accelerometers. The acoustic receiver may be directly positioned in the vicinity of a region of interest on the patient's body or may be carried by an appropriately designed positioning unit. Such an acoustic receiver can be configured to provide an analog electrical output, or may be equipped with an analog-to-digital converter thus providing digital output indicative of the received acoustic waves. The acoustic assembly 12 is preferably a disposable part of the monitoring system, intended for single measurement or to present the so-called “holter monitor” for continuous monitoring.

In operation, the acoustic receiver assembly 12 can be connected to the input of an amplifier (not shown), the output of which can be connected to the control unit 14. It should be understood that amplifier can alternatively be a constructional part of the control unit.

The control unit 14 is typically a computer system having inter alia a memory utility 16 (for storing certain reference data as will be described further below), a data processing and analyzing utility 17, and any data presentation utility such as, for example, a display 18. The data processing and analyzing utility 17 is preprogrammed with a predetermined algorithm for analyzing data indicative of acoustic waves and generating output data about the corresponding urinary system condition.

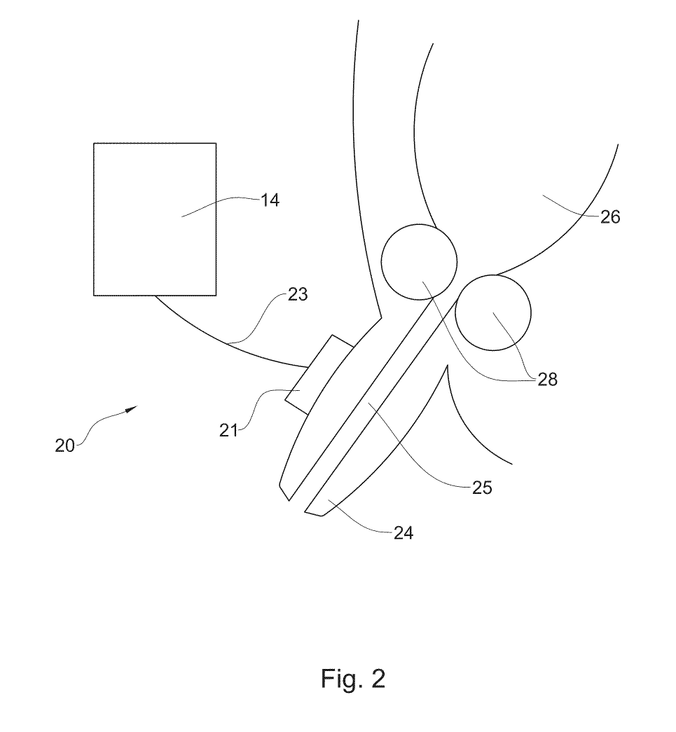

Reference is made to FIG. 2, showing a specific but not limiting example of the configuration and operation of the monitoring system 20 for monitoring a condition of a patient's urinary system. The exemplified system 20 includes an acoustic assembly (12 in FIG. 1), which in the present example of FIG. 2 is formed by a single acoustic receiver 21 (microphone) that is positioned on the patient's body in the vicinity of a region of interest, i.e., in the vicinity of a urine flow region of an urethra 25 in a penis 24. It should be noted that the acoustic assembly may include two or more acoustic receivers, e.g. two such receivers accommodated in a spaced-apart relationship along the region of interest. A control unit 14 is connectable e.g. via wire 23 to the acoustic receiver 21. It should be understood that the acoustic assembly may include more than one acoustic receiver.

The system 20 operates as follows: After the acoustic receiver 21 is held in place, the patient is requested to urinate, and the acoustic receiver 21 continuously receives acoustic waves produced by the urine flow during the urination time. The acoustic receiver output (in the analogue or digital representation) is transmitted to the control unit 14 where the corresponding data indicative of the time variation of the acoustic signal during the urination time is recorded. It should be noted that the acoustic assembly itself may be equipped with an appropriate utility (software and/or hardware) for recording the acoustic data. The control unit 14 operates to process and analyze the acoustic data, to obtain and display information indicative of the urine flow in view of the corresponding condition of the urinary system organs such as the urethra 25 and urinary bladder 26, and a prostate gland 28.

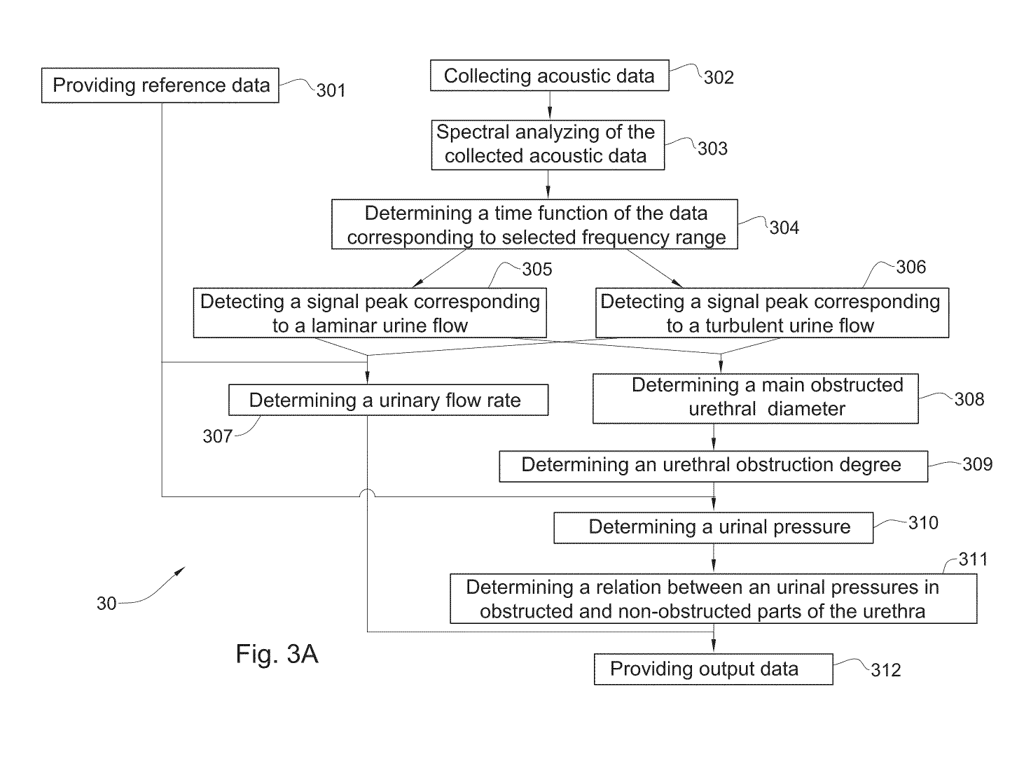

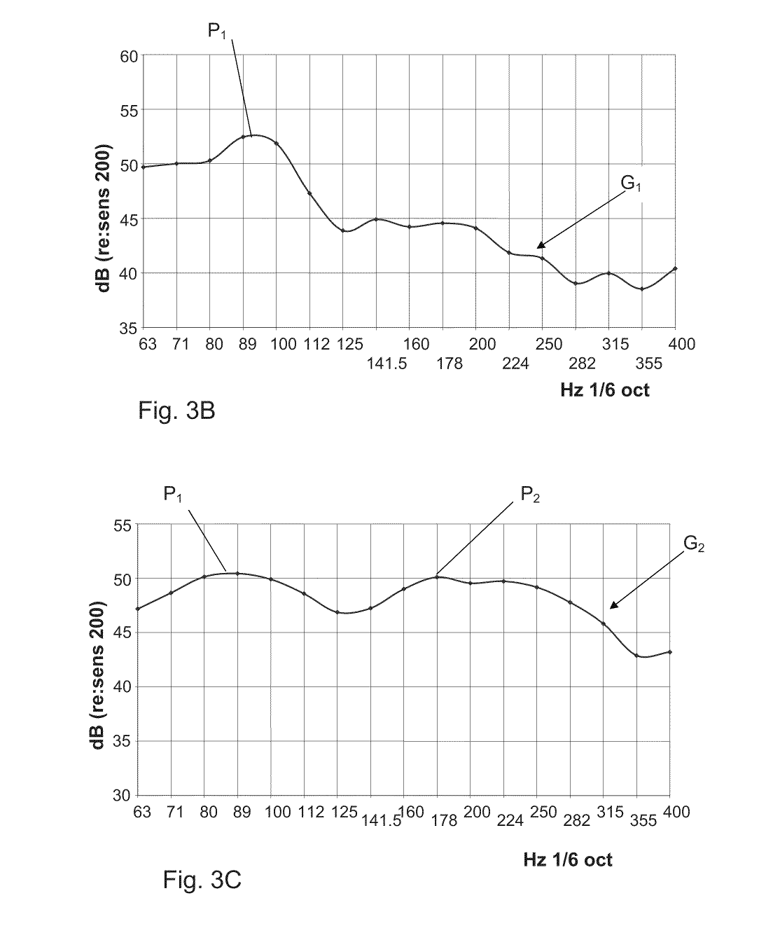

FIG. 3A shows a flow diagram 30 of a method according to an example of the present invention for use in the determination of the condition of a patient's urinary system. As shown in this specific but not limiting example, certain reference data may be provided (step 301) and stored in a memory utility of the control unit. The reference data may include a given value or a range of values for at least one of the following parameters: an urethral diameter, urethral length, and elasticity of an urethral wall previously obtained for the monitored patient or estimated based on the patient's personal data and relevant statistics. The reference data may be obtained by carrying out preliminary measurements. For example, the urethral diameter can be measured using one of X-ray, MRI or various ultrasound methods. Flow velocity can be measured, in particular, by using uroflowmetry, ultrasound based measurements, electromagnetic based measurements or any other technique for measuring urine flow. In addition, the reference data may include relevant data and/or models for healthy patients and patients with various different diseased conditions. Preferably, the reference data includes one or more from the above indicated parameters for different groups of patients, for example of different ages. However, these parameters are known to be varied from individual to individual within small ranges, i.e. with no more than about 15% difference between the lower and upper values of the range.

Acoustic data from a specific patient is collected, either continuously or with a certain sampling model (step 302). This data corresponds to the acoustic waves continuously generated during the patient's urination. In other words, the acoustic data includes the acoustic wave amplitude as a function of time. This may be implemented by segmentation of the sampled data into multiple time windows and performing a Fourier Transform for each such time window, thereby obtaining the acoustic spectral density for each time window and accordingly the time function of the acoustic spectral density.

It should be noted that the acoustic assembly may include multiple acoustic receivers arranged in array(s) along and/or across the region under measurements. In case multiple acoustic receivers are used, such acoustic data may include a single time function from all the receivers, determined by summation or averaging of data received form multiple transducers, or a plurality of such time functions, the entire data thus being a function of coordinate (acoustic receiver location) and time. In this connection, when multiple acoustic receivers are used, data from multiple time functions may be processed using a wavelet transform model (beam forming technique), which enables to locate the obstruction relative to the receivers' array. This can be achieved for example by summing the signals received from all the receivers with different time delays to enhance those signals which originate from particular location and determine the spectral content of said location. When using acoustic data collection from two or more acoustic receivers arranged along the region of interest, determination of the time delay or phase shift between the collected signals may be used for calculating the flow velocity.

The so measured data (time function of acoustic signal) is spectrally analyzed to determine a frequency profile for the received signal (step 303), resulting in the acoustic signal as a function of both, the time and frequency. Specifically, identification of acoustic signals relating to the urination process itself and time-points of the urination initiation and ending can be provided by spectral analysis of the acoustic signals and their intensities. The acoustic spectrum of the urine flow is different from acoustic spectra of other body signals, and thus the urination signals can be detected even in highly rustled conditions.

FIGS. 3B and 3C show the frequency profiles G₁ and G₂ of the so-collected acoustic signal corresponding to respectively a patient with normal condition (non-obstructed) of the urinary system and another patient with a condition of abnormality (obstructed). Both graphs show data corresponding to the 5 sec urination period (average value) being that of the maximal urination rate. As shown, graph G₁ has a well defined signal peak P₁ within a frequency range (about 70-150 Hz) corresponding to the laminar-flow of urine, i.e. at a frequency of about 95 Hz. As shown from graph G₂, in the abnormal condition the laminar-flow related peak P₁ still exists in the respective frequency range and one or more additional peaks appear in the turbulence flow related frequency range (150-1000 Hz), i.e. peak P₂ at about 180 Hz.

In operation, the acoustic signals may be transmitted to the control unit in an analog form and then converted to a digital sequence of amplitude versus time vector or such conversion is implemented in the acoustic assembly (step 304). As indicated above, the signal may be transmitted as an electrical signal via wire or as an RF, IR or acoustic signal via wireless signal transmission. Optionally, such a time function of the acoustic signal can be subject to further signal processing, e.g. an FFT (Fast Fourier Transform) in order to extract frequency and phase from each received signal.

Then, the control unit operates to process and analyze the so-determined spectral data (steps 305, 306). More specifically, this processing is based on the following:

The information relating to the urine flow and relevant processes occurring in the urinary system can be identified and interpreted from the spectral behavior of the acoustic data, including the Strouhal frequency range, which is of about 10-1000 Hz. During the fluid flow in a urethra, a sound is generated with one or more characteristics maxima in its spectrum at a Strouhal frequency, and also outside this range. The Strouhal frequency range, Fₛ, can be expressed by the following relationship:

Fs=Ks·V/D,

where V is a flow velocity, D is an urethral diameter, and Kₛ is the Strouhal Coefficient which has a value of 0.15-0.2 and can be precisely calculated using the Reynolds number which characterizes the flow regime. The Reynolds number appropriate for the urethral flow is estimated as

Re=D·V/ν,

where ν is a dynamic viscosity of the fluid.

The range of Reynolds numbers corresponding to a laminar flow of the fluid along a channel is known as being about 2,000-2,300 (the value of the Strouhal coefficient at these Reynolds numbers is ˜0.1-0.15, while a turbulent flow can be described by Reynolds numbers in a range of about 3,000-30,000. Reynolds numbers in a range of about 2,300-3,000 describe a flow that has features of both laminar and turbulent flows (corresponding to Strouhal coefficient of about 0.2). Acoustic signal peaks at frequencies outside the Strouhal range could also appear in an acoustic signal recorded during the urine flow through the urethra. These peaks are associated inter alia with effects induced by such parameters as urethral length and urethral perimeter on the urine flow and accordingly on the corresponding acoustic waves. For example, relating to the male urethra, the effect of the urethral length corresponds to frequencies above 4 KHz.

The acoustic signal peaks (resonances) caused by effect of elasticity of the pipe's wall onto the urine flow behavior might also be observed most probably be located in a frequency range outside the Strouhal range. The wall elasticity related resonance can be estimated using a spring-mass model with the following parameters: fluid's density, p, that is equal to 1000 Kg/m³, and a tissue's Young's Modulus, E, that is equalized to around 104-105 Pa. With regard to the mass density in the model (that accounts for tissue and fluid mass) it is equal to approximately 2-3 g/cm² (or 20-30 Kg/m²). The relationship between the mass and the spring's elasticity in the model leads to resonant frequencies of a few tens of Hertz, which are slightly dependant on the inner diameter of the pipe.

The actual wall elasticity in the urethra varies to some degree with the advance along the urethra's axis, together with the typical pressures in each cross-section. In the most proximal part of the urethra, i.e. nearest to the bladder outlet, the static pressure is higher than at more distal cross-sections, and the Young's Modulus is also higher. Accordingly, during the urination, the resonant frequency in the corresponding acoustic signal changes along the urethra, and is higher at its beginning and lower at the end. Thus, the acoustic signal amplitude might increase at a certain frequency range with respect to the position along the axis and the static pressure at that cross-section.

Acoustic signals indicative of the urine flow condition are mainly in the Strouhal range. This is because those frequencies associated with other parameters such as urethral length and urethral perimeter are outside the Strouhal range as mentioned above.

Referring back to FIGS. 3A-3C, one or more signal peaks corresponding to condition of a laminar (or laminar-like) urine flow in a frequency range of 70-150 Hz can be detected (step 305). Such laminar urine flow is indicative of the urine flow in non-obstructed parts of the urethra, and would therefore always appear in the received acoustic signals, irrespective of whether the urinary system condition is normal or not. The signals related to the laminar flow can be used, in particular, for analysis of amount of urinated urine and urinal flow velocity, using the above equations.

The urethral obstruction (e.g. by an enlarged prostate) causes a turbulent or turbulent-like urine flow through the urethra, which is of a differing nature than that of laminar urine flow. Such a turbulence flow of the urine generates additional acoustic signals in a frequency range (e.g., 150-1000 Hz) different from that of the laminar flow. Accordingly, the recognition of acoustic signals typical to a turbulent flow is indicative of the urine flow obstruction through the urethra (step 306).

The inventors have found that a relation between the first, laminar flow related peak and the second, turbulent flow related peak (i.e. the frequency and/or magnitude of such peaks in the acoustic signal) is indicative of the obstruction range and of the distance between the acoustic receiver interface and the obstruction's location. This relationship is also indicative of a urinary flow rate. The flow rate can be calculated using reference data (such as the urethral diameter in one or more parts of the urethra, urethral length and elasticity of an urethral wall) (step 307). Also, the flow rate can be estimated from the acoustic measurements: both peaks move towards higher frequencies when the flow becomes to be stronger and move towards lower frequencies when the flow becomes weakly. Accordingly, the spectral analysis preferably covers a frequency range that exceeds the range of 70-150 Hz.

Thus, the relation between the signal peaks indicative of the laminar and turbulent flows can provide data indicative inter alia of main obstruction diameter (step 308). This relation can be calculated as at least one of the following: a ratio between amplitudes of the first and second peaks, a ratio or difference between frequencies of the first and second peaks, and time variations of these ratios/differences during urination and/or during successive urinations.

More specifically, the relation between the urethral part obstructed by the prostate and the unobstructed parts of the urethra by measuring the Strouhal frequencies can be described in the following manner. The Strouhal frequency in the obstructed parts can be calculated by the following relationship:

F₁=0.2·V₁/D₁,

and correspondent Strouhal frequency for unobstructed parts is

F₂=0.2·V₂/D₂.

If the flow volume Q is constant, then

Q=V₁·S₁=V₂·S₂,

where

S=π·D²/4

is the cross sectional area.

Therefore,

V₁·D₁²=V₂·D₂², or V₁/V₂=(D₁/D₂)².

As it was described above, the relation between the frequencies of the first and second peaks can be indicative of a relation between the obstructed and non-obstructed urethral diameters. The following relations can be obtained from previous expressions:

F₁/F₂=C₀=V₁·D₂/V₂·D₁=(V₁/V₂)/(D₁/D₂).

C₀ being the relation between the frequencies of the first and second peaks.

The above relation can be rewritten as following:

V₁/V₂=C₀·D₂/D₁.

Using expression V₁/V₂=(D₁/D₂)² mentioned above, following relation can be obtained:

C₀=(D₂/D₁)³.

or, in other words, the ratio of the diameters in the unobstructed and obstructed parts of the urethra is proportional to the inverse ratio between the first and second Strouhal frequency peaks to the power of three.

Turning back to FIG. 3, the control unit operates to determine the urethral obstruction degree (step 309) using the above ratio of the diameters in the unobstructed and obstructed parts of the urethra. The urethral obstruction degree corresponds to an urethral flow resistance.

Further, the technique of the present invention allows for determining the total value of a urinal pressure in whole urethra as well as in any of its part (step 310).

The urinal pressure, P_{d}, can be calculated by the following relationship:

P_{d}=h·p·g/1000,

where h is a head loss (estimated in meters), p is a fluid density (kg/meters³) and g is a gravitational acceleration (meter/sec²). In its turn, the head loss h can be calculated as follows:

h=f·(L/D)·(V²/2g),

where f is a friction factor, L is an urethral length, D is an urethral diameter, V is a velocity of the fluid (meter/sec) and g is the gravitational acceleration. The urethral length, the urethral diameter and the velocity of the fluid can be obtained from the reference data or preliminary measured by any suitable method. The friction factor can be estimated from Reynolds number which is calculated as described hereinbefore. If Reynolds numbers are less than 2300 (i.e., the urine flow is laminar), the friction factor equals to 64/Re. When the urine flow is turbulent (i.e., Re is higher than 3,000), the friction factor can be calculated by the following relationship:

1/f²=−1.8 log [(6.9/Re)+((k/3.7)^{1.11})],

where k is the relationship between an urethral roughness and the urethral diameter.

Respectively, calculation of a ratio of the urinal pressures in the unobstructed and obstructed parts of the urethra can be performed by using values of the urethral obstruction degree and the reference data such as the urethral diameter, urethral length, and elasticity of an urethral wall (step 311). The pressure profile along the urethra (e.g. measured by an array of acoustic receivers) is indicative of the total urine pressure, which can thus be determined.

The total urine pressure is dependent, inter alia, on amount of the urine in the bladder and on characteristics of the bladder muscles. An effect of each of these parameters on the urinary system condition is associated with the following: the pressure of the muscles is occasional (i.e., during the urination) and the pressure of the urine in the bladder has a continuous feature. Therefore, a possible method to distinguish between these parameters' effects is by measuring the urination during very short time periods, during which an effect of change of the muscle pressure is neglected, but those of a change of the urinary flow rate are significant.

Based on the above processing of the acoustic data in the form of time variation of acoustic signals during the urination time, the present invention provides output data indicative of the urinary system's condition (step 312). The output data can include, but not limited to, the amount of urinated urine (i.e. the integral of urination rate over the urination time), urinal flow velocity, urinary flow rate, urethral obstruction degree, detrusor pressure and pressure in urinary bladder. The output data can be compared to the reference data and the comparison results, being indicative of the existence of physiological abnormalities and the degree of pathology, are displayed to the user, who may be a physician or the patient himself.

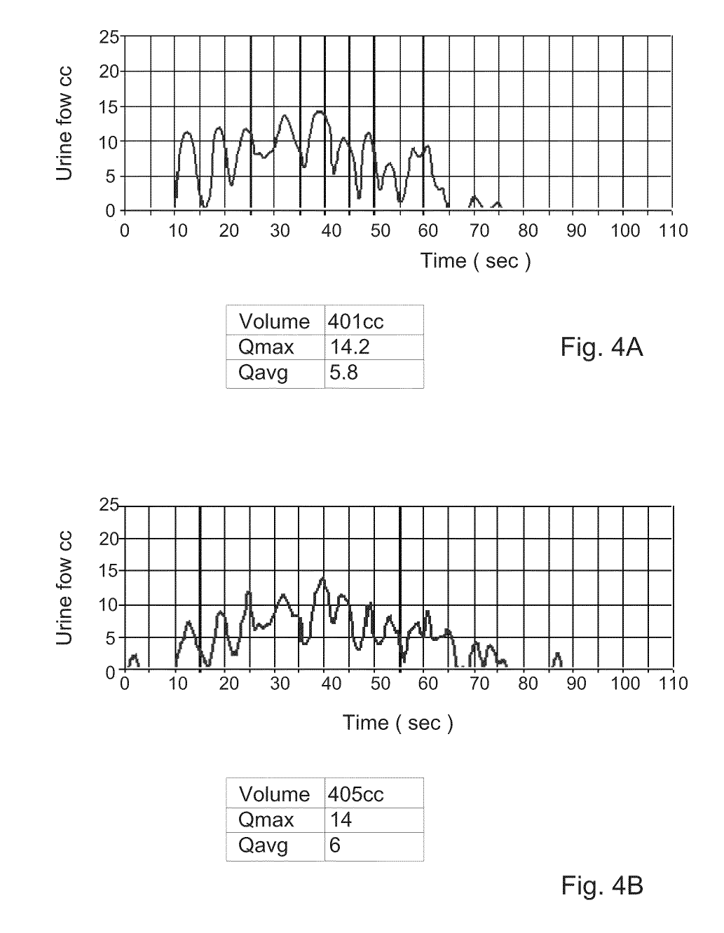

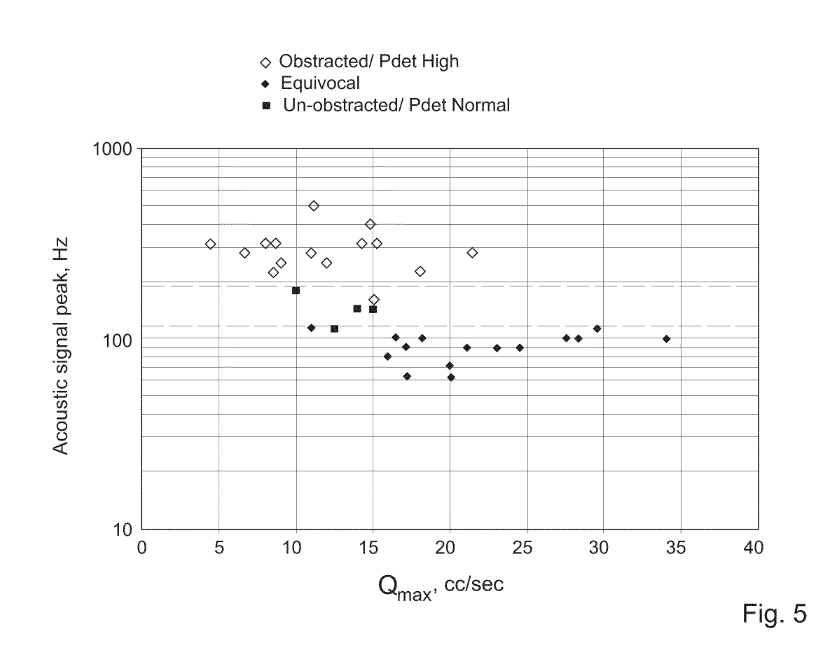

Reference is now made to FIGS. 4-5 showing the experimental results of using the technique of the present invention and corresponding reference methods for examining data indicative of the urinary system's condition.

FIGS. 4A and 4B show quantitative measurements of the urinary flow rate by using, respectively, the acoustic measurements of the present invention and by the commonly used method, i.e. uroflowmeter. As it can be understood from these figures, the urinary flow profile measured according to the method of the present invention and that measured by the uroflowmeter are highly correlated (the graphs are found to be coincident). Moreover, the calculated data of a maximal urinary flow rate (Qₘₐₓ) and an average urinary flow rate (Q_{avg}) are found to be very similar.

FIG. 5 shows graphical representation of data obtained by the technique of the present invention carried out on multiple patients, The figure illustrates the highest acoustic signal peak in a frequency range of 10-1000 Hz at a time point of the maximal urinary flow rate (Qₘₐₓ) as a function of the Qₘₐₓ (calculated in cubic centimeters (or milliliter) per second) in patients with previously diagnosed bladder outlet obstructions and in patients from a control group with no bladder outlet obstructions. In these experiments, 19 patients (ages 34-87, marked by white highlight color) with diagnosed bladder outlet obstructions or with boundary between the normal and obstructed conditions and 15 patients (ages 20-37, marked by black highlight color) from the control group have been examined. As it can be clearly seen from FIG. 5, peak frequencies higher than 200 Hz in the acoustic signals related to the turbulent flow of the urine, indicative of the urine flow obstruction and relatively high detrusor pressure (marked as Pdet high), were found for 14 patients (all with diagnosed bladder outlet obstructions). In addition, peaks of the acoustic signal in a frequency range of 150-200 Hz related to the boundary between the normal and obstructed conditions (i.e. that can equivocally be indicative of the urine flow obstruction) were observed in 4 patients (all diagnosed with boundary between the normal and obstructed conditions). Finally, the existence of only the peak frequencies related to a laminar urine flow (70-150 Hz) and thus to the unobstructed flow and relatively normal/low detrusor pressure (marked as Pdet normal) were found in all 15 patients from the control group.

However, it should be noted that the existing urological approach is to consider the obstructed state in patients if they are characterized by high detrusor or bladder pressure and low urinary flow rate. The technique of the present invention allows for statistical examination of the high detrusor or bladder pressure (i.e., obstructed condition) in patients in which, for example, the determined acoustic peak at a frequency higher than 200 Hz corresponds to Qₘₐₓ that is less than 10 cc/sec. In contrast, non-obstructed (healthy) state can be diagnosed in patients in which 70-150 Hz acoustic peak corresponds to Qₘₐₓ higher than 10 cc/sec.

Those skilled in the art to which the present invention pertains, can appreciate that while the present invention has been described in terms of preferred embodiments, the conception, upon which this disclosure is based, may readily be utilized as a basis for the designing of other structures systems and processes for carrying out the several purposes of the present invention.

In the method claims that follow, alphabetic characters used to designate claim steps are provided for convenience only and do not imply any particular order of performing the steps.

Also, it is to be understood that the phraseology and terminology employed herein are for the purpose of description and should not be regarded as limiting.

Finally, it should be noted that the word “comprising” as used throughout the appended claims is to be interpreted to mean “including but not limited to”.

It is important, therefore, that the scope of the invention is not construed as being limited by the illustrative embodiments set forth herein. Other variations are possible within the scope of the present invention as defined in the appended claims.

1. A monitoring system for use in monitoring a condition of a patient's urinary system, the monitoring system comprising:
(a) an acoustic assembly comprising at least one acoustic receiver, each acoustic receiver being adapted for receiving acoustic signals during a patient's urination and generating data indicative thereof; and
(b) a control unit in communication with said acoustic assembly, the control unit being configured and operable for analyzing the generated data from said at least one acoustic receiver indicative of the received acoustic signals during a patient's urination, determining a time variation of the acoustic signal during the urination and determining a corresponding spectral data of the acoustic signal, analyzing the spectral data and, upon detecting at least one first signal peak corresponding to a condition of turbulence in the urine flow, determining a relation between said first signal peak and a second signal peak corresponding to a condition of laminar urine flow, and based on said relation determining the condition of a patient's low urinary system and generating output data indicative thereof.

2. The system of claim 1, wherein said spectral data includes a Strouhal frequency range.

3. The system of claim 1, wherein said spectral data includes a frequency range of about 20-1000 Hz.

4. The system of claim 1, wherein the second signal peak corresponding to condition of laminar urine flow is in a frequency range of 70-150 Hz.

5. The system of claim 1, wherein the first signal peak corresponding to the turbulent urine flow is in a frequency range of 150-1000 Hz.

6. The system of claim 1, wherein the control unit is configured and operable for analyzing the spectral data by determining a time variation of the relation between the first and second signal peaks, a frequency of at least the first signal peak varying with time during the urination.

7. The system of claim 1, wherein the control unit is configured and operable to determine the relation between the first and second signal peaks by calculating at least one of the following: a ratio between amplitudes of the first and second signals, and a ratio between frequencies of the first and second signals, and time variations of these ratios during urination and/or during successive urinations.

8. The system of claim 1, wherein the control unit comprises a memory utility for storing reference data comprising a given value or a range of values for at least one of the following parameters: an urethral diameter, urethral length, and elasticity of an urethral wall.

9. The system of claim 1, wherein the control unit is configured and operable to apply a predetermined model to the spectral data, said model being based on a given value or a range of values for at least one of the following parameters: an urethral diameter, urethral length, and elasticity of an urethral wall.

10. The system of claim 1, wherein the control unit is configured and operable to process and analyze the relation between the first and second signals or a time variation of the relation between the first and second signals during the urination, and calculate or estimate at least one of the following parameters indicative of the urinary system condition: amount of urinated urine during the urination time; urinal flow velocity profile; urinary flow rate; urethral obstruction degree; pressure in urinary bladder; and detrusor pressure.

11. The system of claim 1, comprising a positioning unit for positioning said at least one acoustic receiver in the vicinity of the patient's urine flow such that an acoustic interface of the receiver is in a position for receiving acoustic signals generated during the patient's urination.

12. A method for use in monitoring a condition of a patient's urinary system, the method comprising:
(a) detecting acoustic signals originated by urine flow during the patient's urination, and generating data indicative thereof;
(b) analyzing said data generated during the urination and determining spectral data indicative thereof;
(c) analyzing the spectral data and, upon detecting at least one first signal peak corresponding to a condition of turbulence in the urine flow, determining a relation between said signal peak corresponding to the condition of turbulence in the urine flow and a second signal peak corresponding to a condition of laminar urine flow, and using said relation to determine the condition of a patient's urinary system and generate output data indicative thereof.

13. The method of claim 12, wherein said detection of the acoustic signals is carried out by at least one acoustic receiver.

14. The method of claim 12, wherein said spectral data includes a Strouhal frequency range.

15. The method of claim 12, wherein said spectral data includes a frequency range of about 20-1000 Hz.

16. The method of claim 12, wherein the signal peak corresponding to a condition of laminar urine flow is in a frequency range of 70-150 Hz.

17. The method of claim 12, wherein the signal peak corresponding to the turbulent urine flow is in a frequency range of 150-1000 Hz.

18. The method of claim 12, wherein said analyzing of the spectral data comprising determining a time variation of the relation between the first and second signal peaks.

19. The method of claim 12, wherein said relation between the first and second signal peaks is indicative of at least one of the following: a ratio between amplitudes of the first and second signals, and a ratio between frequencies of the first and second signals.

20. The method of claim 12, wherein said analyzing of the spectral data comprises applying to said data a predetermined model based on a given value or a range of values for at least one of the following parameters: an urethral diameter, urethral length, and elasticity of an urethral wall.

21. The method of claim 12, wherein said output data indicative of the condition of the urinary system comprises at least one of the following: amount of urinated urine during the urination time, urinal flow velocity profile, urinary flow rate, urethral obstruction degree, urethral flow resistance, pressure in urinary bladder and detrusor pressure.

22. (canceled)

23. A method for use in monitoring a condition of a patient's urinary system, the method comprising: analyzing spectral data corresponding to acoustic signals originated by urine flow during the patient's urination; and upon detecting at least one first signal peak corresponding to a condition of turbulence in the urine flow, determining a relation between said first signal peak and a second signal peak corresponding to a condition of laminar urine flow; and using said relation to determine the condition of a patient's urinary system and generate output data indicative thereof.

24. A computer system adapted for receiving data indicative of a sequence of acoustic signals each corresponding to measurement during a respective urination time, said computer system being configured and operable for processing said data to determine spectral data corresponding to each of the acoustic signals, analyzing the spectral data and, upon detecting at least one first signal peak in the acoustic signal corresponding to a condition of turbulence in the urine flow, determining a relation between said first signal peak and a second signal peak in said acoustic signal corresponding to a condition of laminar urine flow, and based on said relation generating output data indicative of a condition of a patient's urinary system from which said acoustic signals have been originated.

25. The system of claim 1, wherein said acoustic assembly comprises two or two acoustic receivers for accommodation in a spaced-apart relationship along the region of interest, the control unit being configured and operable for analyzing the generated data from each of said two or more acoustic receivers and generating data being a function of time and coordinates of the acoustic receivers with respect to the region of interest.
